(19) United States
(12) Patent Application Publication
(10) Pub. No.: US 2019/0353665 A1
(43) Pub. Date: Nov. 21, 2019

(54) METHOD FOR DIAGNOSING A SKIN DISPLAYING SIGNS OF DRYNESS

(71) Applicant: L'OREAL, Paris (FR)

(72) Inventors: Sandra KANANI, Aulnay-Sous-Bois (FR); Virginie PIFFAUT, Aulnay-Sous-Bois (FR); Aude FOUCHER, Aulnay-Sous-Bois (FR); Nükhet CAVUSOGLU, Aulnay-Sous-Bois (FR); Mark DONOVAN, Aulnay-Sous-Bois (FR)

(21) Appl. No.: 16/466,460

(22) PCT Filed: Dec. 14, 2017
(86) PCT No.: PCT/EP2017/082805

§ 371 Date: Jun. 4, 2019

(30) Foreign Application Priority Data
Dec. 16, 2016 (FR) 16 62670

(51) Int. Cl. G01N 33/68; G01N 33/50; C12Q 1/6883

(52) U.S. Cl. CPC G01N 33/6881; G01N 33/5088; C12Q 2600/158; G01N 2800/20; C12Q 2600/148; C12Q 1/6883

(57) ABSTRACT

The present invention relates to a method for diagnosing a skin displaying signs of dryness, based on the measurement of the level of expression of the gene encoding LCN1. The present invention also relates to a method for the cosmetic treatment of skin. The present invention also relates to a method for identifying a compound suitable for reducing and/or slowing the progression of the signs of dryness of a skin, as well as a kit.

The present invention relates to methods for diagnosing skin dryness.

Various physiological disorders, environmental damage, such as pollution or radiation such as ultraviolet rays, extrinsic factors, such as smoking, variations in the organization, density of the hypodermis, following a modification in body mass, pregnancy, obesity, cellulite, or indeed disorganization of the components of the extracellular matrix, weaken the skin as a whole, which tends to become dry, whether due to a loss of skin hydration or due to a loss of lipids (disruption of the barrier function) giving rise to greater water evaporation and therefore dehydration.

From the above, the importance of finding biomarkers suitable for detecting skin dryness and in particular when said dryness is not yet visible, so as to be able to reduce and/or slow the progression of these signs of skin dryness, is understood. There is a significant need for specific biomarkers of dryness, so as to be able respond in a suitable manner in a given subject.

The aim of the invention is to meet this need.

The applicant discovered surprisingly that the expression of the gene encoding Lipocalin-1 (LCN1), at the level of the skin, is correlated with the presence of signs of skin dryness.

Lipocalin-1 (LCN1) is a small secreted protein of 20 kDa. LCN1 belongs to the calicyn superfamily and the lipocalin family known to bind with and transport small lipophilic molecules such as retinol and retinoic acid.

As such, according to a first of the aspects thereof, the invention relates to a method for diagnosing, preferentially in vitro, a skin displaying signs of dryness, in a subject, said method comprising the following steps:

a) measuring the level of expression of the gene encoding LCN1, in particular the level of protein expression of the gene encoding LCN1, in a skin sample from said subject, and

b) optionally, on the basis of the level of expression of the gene encoding LCN1 measured in step a), determining whether said subject's skin displays signs of dryness.

The present invention also relates to a method for the cosmetic treatment of a skin displaying signs of dryness in a subject, said method comprising the following steps:

a) measuring the level of expression of the gene encoding LCN1, in a skin sample from said subject;

b) inferring from step a) whether said subject's skin displays signs of dryness;

c) if the skin is identified as displaying signs of dryness in step b), treating said skin with a cosmetic composition suitable for reducing and/or slowing the progression of the signs of skin dryness.

In the context of the skin, the term skin “dryness” covers both skin dehydration (water loss) and a loss of lipids from the skin (generally in turn giving rise to dehydration).

The term “signs of skin dryness” denotes herein any skin modifications arising with the loss of water and/or lipids from the skin and which are sometimes not visible at the early stages, and in particular sensations of tautness and a rough texture. In particular, dryness of the skin of the face and preferably of the cheeks is referred to herein.

LCN1 is a protein known in a plurality of isoforms wherein the amino acid sequences are known and particularly the sequence having the following GenBank accession code: AAH74926.1 (version dated Sep. 23, 2014). The mRNA sequence encoding LCN1 is also known to those skilled in the art and particularly accessible with the NCBI accession code: NM_002297.3 (version dated Jun. 12, 2016).

The term “level of expression of the gene encoding LCN1” denotes the level of expression of messenger RNA (mRNA) of the gene encoding LCN1 and/or the level of expression of LCN1 protein.

Preferentially, the level of expression of LCN1 protein is measured using a specific ligand of LCN1 protein, such as for example an antibody, preferably monoclonal, a Fab fragment, an scFv or a nanobody, specific for this protein. The level of expression may then be measured by means of any method known to those skilled in the art, such as for example by means of an ELISA test, preferably a sandwich ELISA using a capture antibody, specific for LCN1 protein and a detection antibody, also specific for LCN1 protein. In particular, the measurement of the level of expression of LCN1 protein may be performed using a miniaturized system such as a microfluidic chip, such as that described in the international application WO2011/126249, this chip containing specific ligands for LCN1 protein. The chip may subsequently be analyzed by means of a suitable reader in order to determine whether LCN1 proteins have bound with said ligand and reach a conclusion in respect to the presence and quantity of LCN1 in the skin sample.

Preferentially, the level of expression of mRNA of the gene encoding LCN1 is measured using a complementary nucleotide sequence of the mRNA of the gene encoding LCN1 and specifically hybridizing with the mRNA of the gene encoding LCN1 or, a fragment thereof hybridizing specifically with the mRNA of the gene encoding LCN1, this sequence or this fragment comprising 5 to 50 nucleotides, preferentially 10 to 20 nucleotides, or using a pair of primers or a probe of 10 to 60 nucleotides, preferentially 15 to 30 nucleotides comprising said sequence or said fragment. The level of expression may then be measured by any means known to those skilled in the art, for example by means of quantitative PCR.

Within the scope of the invention, the terms “hybridize” or “hybridization”, as well-known to those skilled in the art, refer to the bonding of a nucleic acid sequence with a particular nucleotide sequence under suitable conditions, particularly under stringent conditions.

The term “stringent conditions”, as used herein, corresponds to conditions which are suitable for producing bond pairs between the nucleic acids having a defined level of complementarity, while being unsuitable for the formation of pairs between the bonding nucleic acids having a lower complementarity than said defined level. The stringent conditions are dependent on hybridization and washing conditions. These conditions may be modified according to methods known to those skilled in the art. Generally, high-stringency conditions are a hybridization temperature approximately 5° C. less than the melting point (Tm), preferably close to the Tm of the perfectly base-paired strands. The hybridization procedures are well-known in the art.

High-stringency conditions generally involve hybridization at a temperature of approximately 50° C. to approximately 68° C. in a 5×SSC/5× Denhardt's solution/1.0% SDS solution, and washing in a 0.2×SSC/0.1% SDS solution at a temperature between approximately 60° C. and approximately 68° C.

According to one preferred embodiment, the skin sample of the subject used in the diagnostic and cosmetic treatment methods according to the invention, is a sample taken, preferably non-invasively, on the subject's skin, preferentially on the subject's face, in particularly on the subject's cheek. Preferentially, the skin sample is obtained from the stratum corneum. Preferably the skin sample is a sample taken on the subject's skin in an area without lesions.

The stratum corneum is the outermost layer of the epidermis, and comprises the skin surface. It is essentially made up of dead cells.

According to one embodiment, the methods according to the invention comprise a step for taking the skin sample from the subject. This step is preferentially performed non-invasively, and in particular does not require local anesthetic. According to one preferred embodiment, the step for taking the sample is performed by rubbing the skin surface or using an adhesive surface such as a D-squame® disc.

The term “subject” denotes a human being, preferably aged from 20 to 90 years, preferentially from 35 to 80 years, from 45 to 80 years and even more preferentially from 60 to 80 years. Preferably, the subject is female. Preferentially, the subject does not suffer from dermatologic lesion and/or from dermatologic disease.

In a particular embodiment, the reference value is between 50 and 90 ng/ml of LCN1 protein, preferably the reference value is between 60 and 80 ng/ml of LCN1 protein and more preferably the reference value is between 67 and 75 ng/ml of LCN1 protein.

In one particular embodiment, step (b) is carried out after comparing the level of expression after comparing the level of expression of the gene encoding LCN1 obtained with a reference value. In one embodiment, the reference value is determined by the mean value of the level of expression of the gene encoding LCN1 in a defined population, for example a population in a defined age-group and/or having a defined skin type (Caucasian, Asian, etc.). In one particular embodiment, the reference value is determined by the mean value of the level of expression of the gene encoding LCN1 in women aged from 60 to 80 years. In a further embodiment, the reference value is the optimal threshold value determined by ROC analysis. According to the invention, a reference value may be determined by a plurality of samples, preferably more than 50, 100, 200 , 300 or 500 samples.

The term “comparison” refers to determining whether the level of expression of the gene encoding LCN1 is essentially identical to a reference value or differs therefrom. Preferably, the level of expression of the gene encoding LCN1 is considered to be different from a reference value if the difference observed is statistically significant. If the difference is not statistically significant, the level of expression of the gene encoding LCN1 and the reference value are essentially identical.

On the basis of this comparison, it is possible to determine whether the skin displays signs of dryness. Typically, the skin is considered to display signs of dryness when the level of expression of the gene encoding LCN1 is significantly lower than the reference value and the skin is not considered to display signs of dryness when the level of expression of the gene encoding LCN1 is essentially identical or significantly greater than the reference value.

The term “cosmetic composition suitable for reducing and/or slowing the progression of the signs of skin dryness” denotes any cosmetic composition known to those skilled in the art suitable for reducing and/or slowing the progression of the signs of skin dryness.

The invention also relates to a method for identifying a compound suitable for reducing and/or slowing the progression of the signs of dryness of a skin, said method comprising the following steps:

a) measuring the level of expression of the gene encoding LCN1, in a skin sample exposed to said candidate compound;

b) comparing said level of expression to the level of expression in a sample of said skin not exposed to said compound;

c) identifying said candidate compound as a compound suitable for reducing and/or slowing the progression of the signs of dryness of a skin when an increase in the level of expression of the gene encoding LCN1 in the skin sample exposed to said candidate compound is detected, with respect to the level of expression of the gene encoding LCN1 in the skin sample not exposed to the candidate compound.

According to one preferred embodiment, the skin sample used in the method for identifying a compound suitable for reducing and/or slowing the progression of the signs of skin aging according to the invention is a skin cell culture, an epidermis culture, a reconstructed whole skin, a human skin explant, or a skin sample from a subject as defined above.

Preferentially, the methods according to the invention comprise a step for treating the sample so as to prepare the measurement of the level of expression of the gene encoding LCN1. For example, the treated sample is a soluble protein extract. In particular, if the skin sample has been obtained using an adhesive surface such as a D-squame® disc, the soluble protein extract may be obtained using a protein extraction system such as that described in the international application WO2015/009085 which is suitable for contacting the D-squame samples with a minimal volume of extraction buffer and thereby obtaining a soluble protein extract.

The present invention also relates to a kit comprising:

a) means for measuring the expression of the gene encoding LCN1 in a skin sample, and

b) an instruction leaflet.

The term “means for measuring the expression of the gene encoding LCN1” denotes a specific ligand of LCN1 protein, such as for example an antibody, preferably monoclonal, a Fab fragment, an scFv or a nanobody, specific for this protein or a microfluidic chip such as that described in the international application WO2011/126249, this chip containing specific ligands for LCN1 protein. Alternatively, a complementary nucleotide sequence of the mRNA of the gene encoding LCN1 and specifically hybridizing with the mRNA of the gene encoding LCN1 or, a fragment thereof hybridizing specifically with the mRNA of the gene encoding LCN1 is denoted, this sequence or this fragment comprising 5 to 50 nucleotides, preferentially 10 to 20 nucleotides, or a pair of primers or a probe of 10 to 60 nucleotides, preferentially 15 to 30 nucleotides comprising said sequence or said fragment.

Preferably, the means for measuring the expression of the gene encoding LCN1 is at least one specific ligand of LCN1, in particular a pair of capture and detection antibodies specific for LCN1 protein.

According to one embodiment, the kit according to the invention further comprises means for taking the skin sample such as a D-squame® disc.

The present invention further relates to the use of a kit according to the invention for identifying a compound suitable for reducing and/or slowing the progression of the signs of dryness of a skin.

The present invention will be illustrated by the following example.

### EXAMPLE

The level of visible skin dryness was evaluated on the cheeks of 376 women aged 36 to 75 years. The level of protein expression of the gene encoding LCN1 was also evaluated on these women's cheeks, by means of a D-squame sample wherein the soluble protein extract was analyzed using an ELISA test conducted on a chip.

The univariate statistical analysis of the results obtained shows a correlation between the presence of these signs of dryness and the expression of LCN1 protein.

TABLE 1

| | | | | threshold |
|---|---|---|---|---|
| | | | | value (in |
| clinical signs | accuracy | sensitivity | specificity | ng/ml) |
| dryness | 49% | 69% | 43% | 72.23 |

1. A method for diagnosing a skin displaying signs of dryness, in a subject, said method comprising measuring the level of expression of the gene encoding LCN1, in a skin sample from said subject.

2. A method for the cosmetic treatment of a skin displaying signs of dryness in a subject, said method comprising the following:
a) measuring the level of expression of the gene encoding LCN1, in a skin sample from said subject;
b) inferring from step a) whether said subject's skin displays signs of dryness;
c) if the skin is identified as displaying signs of dryness in step b), treating said skin with a cosmetic composition suitable for reducing and/or slowing the progression of the signs of skin dryness.

3. A method for identifying a compound suitable for reducing and/or slowing the progression of the signs of dryness of a skin, said method comprising the following:
a) measuring the level of expression of the gene encoding LCN1, in a skin sample exposed to a candidate compound;
b) comparing said level of expression measured in step a) to the level of expression of the gene encoding LCN1 in a skin sample not exposed to said compound;
c) identifying said candidate compound as a compound suitable for reducing and/or slowing the progression of the signs of dryness of a skin when an increase in the level of expression of the gene encoding LCN1 in the skin sample exposed to said candidate compound is detected, with respect to the level of expression of the gene encoding LCN1 in the skin sample not exposed to the candidate compound.

4. The method according to claim 3, wherein said skin sample is a skin cell culture, an epidermis culture, a reconstructed whole skin, a human skin explant, or a skin sample from a subject.

5. The method according to claim 1, wherein the skin sample is taken using a D-squame® disc.

6. The method according to claim 1, wherein the level of expression of the gene encoding LCN1 is determined by the level of expression of mRNA of said gene or by measuring the level of expression of the protein encoded by said gene, preferably by measuring the level of expression of the protein encoded by said gene.

7. The method according to claim 6, wherein the level of expression of the protein encoded by the gene encoding LCN1 is measured using at least one specific ligand of LCN1 protein.

8. A kit comprising:
a) means for detecting, in a skin sample, the expression of the gene encoding LCN1, and
b) an instruction leaflet
c) means for taking a skin sample.

9. The kit according to claim 8 wherein the means for detecting, in a skin sample, the expression of the gene encoding LCN1 is at least a specific ligand of LCN1, preferably a pair of capture and detection antibodies specific for LCN1 protein.

10. A method for identifying a compound suitable for reducing and/or slowing the progression of the signs of dryness of a skin which comprises using the kit according to claim 8.

11. A method for identifying a compound suitable for reducing and/or slowing the progression of the signs of dryness of a skin which comprises using the kit according to claim 9.

12. The method according to claim 2, wherein the skin sample is taken using a D-squame® disc.

13. The method according to claim 3, wherein the skin sample is taken using a D-squame® disc.

14. The method according to claim 4, wherein the skin sample is taken using a D-squame® disc.

15. The method according to claim 2, wherein the level of expression of the gene encoding LCN1 is determined by the level of expression of mRNA of said gene or by measuring the level of expression of the protein encoded by said gene, preferably by measuring the level of expression of the protein encoded by said gene.

16. The method according to claim 3, wherein the level of expression of the gene encoding LCN1 is determined by the level of expression of mRNA of said gene or by measuring the level of expression of the protein encoded by said gene, preferably by measuring the level of expression of the protein encoded by said gene.

17. The method according to claim 4, wherein the level of expression of the gene encoding LCN1 is determined by the level of expression of mRNA of said gene or by measuring the level of expression of the protein encoded by said gene, preferably by measuring the level of expression of the protein encoded by said gene.

18. The method according to claim 5, wherein the level of expression of the gene encoding LCN1 is determined by the level of expression of mRNA of said gene or by measuring the level of expression of the protein encoded by said gene, preferably by measuring the level of expression of the protein encoded by said gene.

19. The method according to claim 15, wherein the level of expression of the protein encoded by the gene encoding LCN1 is measured using at least one specific ligand of LCN1 protein.

20. The method according to claim 16, wherein the level of expression of the protein encoded by the gene encoding LCN1 is measured using at least one specific ligand of LCN1 protein.
